(19) United States
(12) Patent Application Publication
(10) Pub. No.: US 2010/0215585 A1
(43) Pub. Date: Aug. 26, 2010

(54) DYES AND PRECURSORS AND CONJUGATES THEREOF

(76) Inventor: John V. Frangioni, Wayland, MA (US)

Correspondence Address: FISH & RICHARDSON PC, P.O. BOX 1022, MINNEAPOLIS, MN, 55440-1022, US

(21) Appl. No.: 12/376,243

(22) PCT Filed: Aug. 3, 2007
(86) PCT No.: PCT/US07/75216

§ 371 Date: Apr. 13, 2010

Related U.S. Application Data
(60) Provisional application No. 60/835,344, filed on Aug. 3, 2006.
(60) Provisional application No. 60/835,407, filed on Aug. 3, 2006.

(51) Int. Cl. A61K 49/00; C07D 209/04; A61P 43/00

(52) U.S. Cl. 424/9.6; 548/455

(57) ABSTRACT

Novel dyes, precursors to novel dyes, and conjugates of the novel dyes are disclosed, as well as methods of making and using the same.

### CROSS-REFERENCE TO RELATED APPLICATIONS

This application claims priority from U.S. Provisional Application Ser. No. 60/835,344, filed on Aug. 3, 2006, and U.S. Provisional Application Ser. No. 60/835,407, filed on Aug. 3, 2006, the contents of which are incorporated herein by reference in their entireties.

### TECHNICAL FIELD

This invention relates to dyes, and to precursors and conjugates thereof.

### BACKGROUND

Generally, cyanine dyes have a delocalized electron system that spans over many carbon atoms. FIG. 1 shows one, such dye, 2-(2-[2-chloro-3-([1,3-dihydro-1,3,3-trimethyl-2H-indol-2-ylidene]ethylidene)-1-cyclohexen-1-]ethenyl)-1,3,3-trimethylindolium iodide, which is commonly known as IR-786 (1)A. The synthesis of some cyanine dyes is described in Little et al., U.S. Pat. No. 6,027,709; Lugadc et al., U.S. Pat. No. 6,995,274, and U.S. Patent Application Publication No. 2006/0063247; Achilefu et al., U.S. Pat. No. 6,939,532; and Li et al., Synthesis and Characterization of Heptamethine Cyanine Dyes, Molecules, 2, 91-98 (1997).

Cyanine dyes, which often have an intense absorption and emission in the near-infrared (NIR) region, can be useful for biomedical fluorescence imaging because biological tissues are typically optically transparent in this region. Several studies on the use of NIR dyes, and dye-biomolecule conjugates have been published. For example, see Patonay et al., Near-Infrared Fluorogenic Labels: New Approach to an Old Problem, Analytical Chemistry, 63:321A-327A (1991); Brinkley, A Brief Survey of Methods for Preparing Protein Conjugates with Dyes, Haptens, and Cross-Linking Reagents, Perspectives in Bioconjugate Chemistry, pp. 59-70, C. Meares (Ed), ACS Publication, Washington, D.C. (1993); Slavik, Fluorescent Probes in Cellular and Molecular Biology, CRC Press, Inc. (1994); Lee et al., U.S. Pat. No. 5,453,505; Hohenschuh et al., WO 98/48846; Turner et al., WO 98/22146; Kai et al., WO 96/17628; Snow et al., WO 98/48838; and Frangioni et al., IRDye78 Conjugates for Near-Infrared Fluorescence Imaging, Molecular Imaging, 1(4):354-364 (2002).

### SUMMARY

Generally, the new dyes and conjugates described herein have non-ionic solubilizing arms, which can effectively “shroud” the positive charge on the dye nucleus, reducing the overall effective charge of the molecule. This shrouding dramatically enhances the stability of the dyes, and conjugates, and their solubility in biological fluids. The enhanced solubility and stability of the new dyes and conjugates reduces non-specific background noise during surgery. In addition, the increased solubility enables the use of these new dyes in many biological applications.

As used herein, non-ionic solubilizing arms are neutral moieties, such as oligomers or polymers, that are capable of interacting strongly with, e.g., capable of forming hydrogen bonds with, water. Examples include polyethylene glycols (PEGs), polypropylene glycols, or copolymers of polyethylene oxide, and polypropylene oxide. For these specific examples, each oxygen atom on the molecular arm can interact strongly with a molecule of water.

More particularly, some of the dyes disclosed herein include a positively charged nitrogen-containing dye core that includes a conjugated heptamethine or substituted heptamethine system. As used herein, “a heptamethine system” is an uninterrupted molecular fragment that includes seven methine groups (CH groups), and having a delocalized electron density, whereas a substituted heptamethine system is the same, but with one or more of the hydrogen atoms substituted with other groups. The dye core has one or more non-ionic solubilizing molecular'arms and, optionally, one or more functionalizable molecular arms bonded thereto. When present, the one or more functionalizable molecular arms can include an amine-, alcohol-, or thiol-reactive carboxylic acid group, anhydride group, ester group, or isothiocyanate group. As used herein, “a functionalizable molecular arm” is a moiety that can be conjugated. For example, the molecular arm can be conjugated with a protein, or a carbohydrate. The dye core can include a single positive charge, or multiple charges.

The dyes have a high solubility in vitro, and in biological systems. For example, the one or more solubilizing molecular arms can be selected such that the dyes have a solubility in 10 mM HEPES solution (N-(2-hydroxyethyl)piperazine-N′-(2-ethanesulfonic acid)), pH 7.4, of greater than about 10 μM, e.g., greater than 25, 50, 75, 100, 125, 150, or even greater than 250 μM. If desired, the one or more solubilizing arms can also be functionalized with an amine-, alcohol-, or thiol-reactive carboxylic acid group, anhydride group, ester group, or isothiocyanate group. Generally, the dyes have an intense absorption and/or emission at a wavelength of from about 300 nm to 1000 nm, and thus emit in the green, yellow, orange, red, and near infrared portions of the spectrum. For example, the dyes can have a maximum excitation and/or a maximum emission, measured in 10 mM HEPES solution, pH 7.4, of from about 525 nm to about 875 nm, e.g., from about 550 nm to about 825 nm, or from about 550 nm to about 800 nm.

Some dyes are described that include cations represented by Structure (1), which is shown below. In general, such cations include a substituted heptamethine system and have solubilizing molecular arms in at least four positions, represented by S₁, S₂, S₃, and S₄. Also, in general, such cations include a fifth molecular arm, represented by G in Structure (I). G can be, or can include, e.g., an amine, alcohol- or thiol-reactive carboxylic acid group, anhydride group, ester group, or isothiocyanate group, or a solubilizing molecular arm, e.g., a polyethylene glycol, e.g., one terminated with a hydroxyl group. Optionally, a portion of the fifth molecular arm can include a solubilizing moiety, such as a polyethylene glycol spacer. Conjugates can be formed by reacting the fifth molecular arm (or any of the other arms) with an amino-, hydroxyl-, or thiol-containing moiety, such as a small molecule peptide, a protein, a polypeptide, or a carbohydrate.

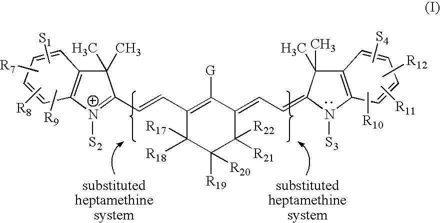

In one aspect, the invention features compounds that include cations of Structure (I), in which S₁, S₂, S₃, and S₄ are each independently a non-ionic oligomeric or polymeric solubilizing moiety; G is H, a moiety that includes at least one amine, alcohol- or thiol-reactive carboxylic acid group, anhydride group, ester group, or isothiocyanate group, or a non-ionic oligomeric or polymeric solubilizing moiety; R₇, R₈, R₉, R₁₀, R₁₁, R₁₂, R₁₇, R₁₈R₁₉, R₂₀, R₂₁, and R₂₂ are each independently H, F, Cl, Br, I, C1-C6 straight-chain or branched alkyl, C1-C6 straight-chain or branched alkoxy, an aromatic ring having up to 6 carbon atoms, optionally substituted with one or more of F, Cl, Br, or I, or any two or more of R₇, R₈ and R₉; R₁₀, R₁₁ and R₁₂; and/or R₁₇, R₁₈, R₁₉, R₂₀, R₂₁, and R₂₂ may be bonded together to define a ring that includes between 5 and 12 carbon atoms. The ring that includes between 5 and 12 carbon atoms is optionally substituted with one or more F, Cl, Br, or I.

In some embodiments, S₁-S₄ are selected such that compounds that include cations of Structure (I) have a solubility in 10 mM HEPES solution, pH 7.4, of greater than about 10 μM.

In another aspect, the invention features compounds that include cations of Structure (XV)

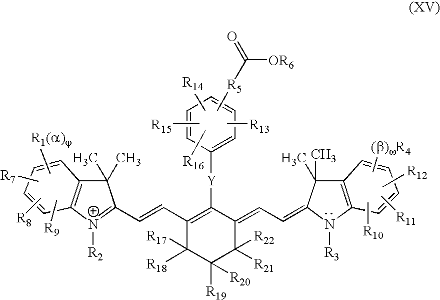

in which φ and ω are each independently 0 or 1; α and β are each independently O, S, CH₂, CH₂O, CO₂, or NR' in which R′ is H or C1-C6 straight-chain or branched alkyl; R₁, R₂, R₃, and R₄ are each independently (CH₂CH₂O)ₙR″ in which R″ is H or C1-C6 straight-chain or branched alkyl, n being an integer from 4 to 2,500; Y is S or O; R₅ is (CH₂)ₘ, m being an integer from 0 to 8 or a non-ionic oligomeric or polymeric solubilizing moiety; R₆ is H, C1-C6 straight-chain or branched alkyl, or N-succinimidyl; R₇, R₈, R₉, R₁₀, R₁₁, R₁₂, R₁₇, R₁₈, R₁₉, R₂₀, R₂₁, and R₂₂ are as described above in reference to Structure (I); and R₁₃, R₁₄, R₁₅, and R₁₆ are each independently H, F, Cl, Br, I, C1-C6 straight-chain or branched alkyl, C1-C6 straight-chain or branched alkoxy, or an aromatic ring having up to 6 carbon atoms, optionally substituted with one or more F, CI, Br, or I.

In some embodiments, R₁-R₄ are selected such that compounds that include cations of Structure (XV) have a solubility in 10 mM HEPES solution, pH 7.4, of greater than about 10 μM.

In another aspect, the invention features compounds of Structure (V)

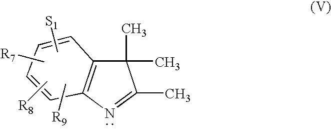

in which S₁, and R₇, R₈, and R₉ are as described above in reference to Structure (I).

In some embodiments, S₁ is selected such that compounds of Structure (V) have a solubility in 10 mM HEPES solution, pH 7.4, of greater than about 10 μM.

In another aspect, the invention features compounds that include cations of Structure (VI)

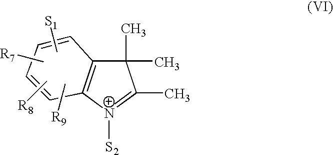

in which S₁, S₂, R₇, R₈, and R₉ are as described above in reference to Structure (I).

In some embodiments, S₁, and S₂ are selected such that compounds that include cations of Structure (VI) have a solubility in 10 mM HEPES solution, pH 7.4, of greater than about 10 μM.

In another aspect, the invention features compounds that include cations of Structure (VIII)

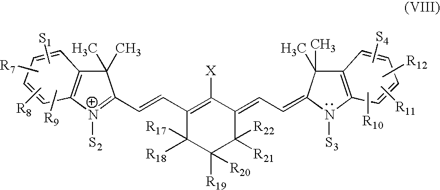

in which S₁, S₂, S₃, S₄, R₇, R₈, R₉, R₁₀, R₁₁, R₁₂, R₁₇, R₁₈, R₁₉, R₂₀, R₂₁, and R₂₂ are as described above in reference to Structure (I); and X is Cl, Br, I, or tosylate.

In some embodiments, S₁—S₄ are selected such that compounds that include cations of Structure (VIII) have a solubility in 10 nM HEPES solution, pH 7.4, of greater than about 10 μM.

Aspects and/or embodiments of the invention can have any one of, or combinations of, any of the following advantages. The dye precursors, dyes, and conjugates have a high solubility in aqueous solutions, and biological fluids and tissues. The dyes and conjugates have non-ionic solubilizing arms, which can effectively “shroud” the positive charge on the nitrogen atoms, reducing the overall effective charge of the molecule. Reducing the overall effective charge can minimize non-specific background noise during imaging. The dyes and conjugates can be used for real time surgical guidance for identifying tumors and other abnormal tissues. The dyes and conjugates generally have a high in vivo stability. The dyes are easily conjugated with targeting molecules, such as those that contain amino, thiol, and/or hydroxyl functionality. The dyes and conjugates retain high fluorescent yield at about 800 nm, which is often optimal for in vivo imaging. Solubilizing arms on the dyes and conjugates have a length that can be adjusted to optimize biodistribution and clearance. The solubilizing arms of the dyes and conjugates can reduce non-specific background binding in vivo. The dyes and conjugates can have a low overall toxicity.

For the purposes of this disclosure, 10 mM HEPES solution, pH 7.4, is, a pH adjusted, 10 mM solution of N-(2-hydroxyethyl)piperazine-N′-(2-ethanesulfonic acid).

For mixtures of materials, such as mixtures of monomeric compounds or polymeric compounds that have a molecular weight distribution, solubility is the average solubility of dye core.

An “oligomer” as used herein, is a relatively low molecular weight polymer having between about 4 and about 25 repeat units.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference herein in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a resonance structure for 2-(2-[2-chloro-3-([1,3-dihydro-1,3,3-trimethyl-2H-1-indol-2-ylidene]ethylidene)-1-cyclohexen-1-yl]ethenyl)-1,3,3-trimethylindolium iodide (IR-786, (1)A).

FIG. 2A is a generalized reaction scheme, illustrating attachment of solubilizing arms onto functionalized anilines.

FIG. 2B is a representation of structures of specific functionalized anilines, and corresponding anilines having attached solubilizing arms.

FIG. 3 is a generalized reaction scheme, illustrating preparation of diazonium salts (not shown) corresponding to the anilines of FIG. 2A having the solubilizing arms, and then reduction of the diazonium salts to produce the corresponding hydrazines.

FIG. 4 is a generalized reaction scheme, illustrating cyclization of the hydrazines of FIG. 3, utilizing methyl isopropyl ketone and the Fischer indole reaction.

FIG. 5 is a generalized reaction scheme, illustrating quaternization of the cyclized products of FIG. 4.

FIG. 6A is a generalized reaction scheme, illustrating coupling of the quaternized products of FIG. 5 to produce intermediate dyes.

FIG. 6B is a representation of three structures of several specific hydroxyl methylene cyclohexenes.

FIG. 7A is a generalized reaction scheme, illustrating, producing secondary dyes from the intermediate dyes of FIG. 6A.

FIG. 7B is a representation of three structures of specific G′ reactants that can react with compounds of Structure (VIII)A of FIG. 7A to produce compounds of Structure (I)A of FIG. 7A.

FIGS. 8 and 9 are general reaction schemes, illustrating alternative synthetic pathways to produce dye precursor components.

FIG. 10 is a general reaction scheme, illustrating alternative synthetic pathways.

FIG. 11 is a reaction scheme, illustrating preparation of hydrazines from hydroxyanilines; cyclization of the hydrazines using methyl isopropyl ketone and the Fischer indole reaction; and then attaching PEG solubilizing arms onto the functionalized cyclized products and quaternization of the cyclized products.

FIG. 12 is a reaction scheme, illustrating coupling of the quaternized products of FIG. 11 to produce intermediate dyes.

FIG. 13 is a reaction scheme, illustrating preparation of secondary dyes from the intermediate dyes of FIG. 12.

FIG. 14 is a reaction scheme, illustrating preparation of an m-methoxy phenyl hydrazine from m-methoxy aniline; cyclization of the hydrazine using methyl isopropyl ketone and the Fischer indole reaction; metalating the ortho to the methoxy group and heterocyclic ring to produce a carbanion (not shown); and then attaching PEG solubilizing arms onto the functionalized cyclized products and quaternization of the cyclized products.

FIG. 15 is a generalized reaction scheme showing the preparation of a conjugate from a dye, and a hydroxyl-containing moiety, e.g., a carbohydrate.

FIG. 16 is a generalized reaction scheme, illustrating the preparation of a conjugate from a dye, and an amino-containing moiety, e.g., a protein.

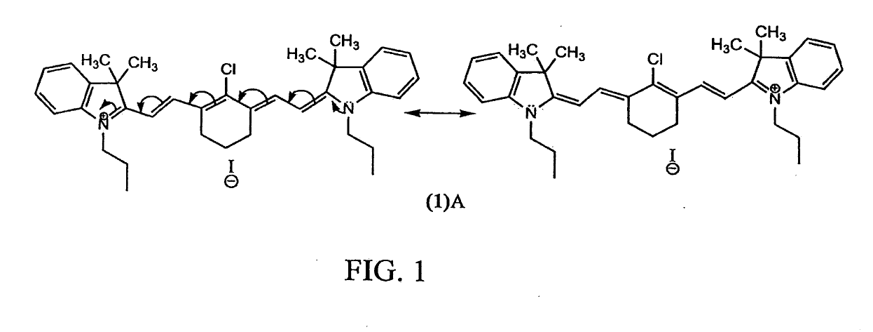

### DETAILED DESCRIPTION

Dyes are provided that include non-ionic solubilizing moieties, such as polyethylene glycols (PEG). Such dyes can be conjugated, e.g., by reacting the dyes with a protein or a carbohydrate, to provide imaging agents that can bind selectively to certain tissues, e.g., abnormal tissues, allowing for their imaging. For example, dyes and conjugates can be used for real time surgical guidance for identifying tumors, and other abnormal tissues.

### Dyes

Some dyes are provided that include cations represented by Structure (I), which is shown below.

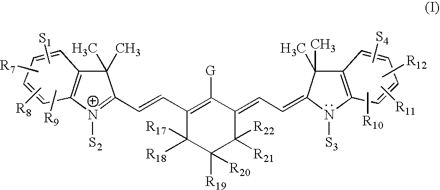

In dyes that include cations of Structure (I), S₁, S₂, S₃, and S₄ are each independently a non-ionic oligomeric or polymeric solubilizing moiety.

In some embodiments, S₁-S₄ are selected such that the dyes that include the cations of Structure (I) have a solubility in 10 mM HEPES solution (N-(2-hydroxyethyl)piperazine-N′-(2-ethanesulfonic acid)), pH 7.4, of greater than about 10 μM, e.g., greater than 25, 50, 75, 100, 125, 150, 200, or even greater than 250 μM. Solubility can be determined photometrically at 25° C. by setting up a calibration curve using a base dye core; saturating a 10 mM HEPES solution, pH 7.4, with the test compound or mixture, and then determining where on the calibration curve the test compound or mixture falls.

For example, each non-ionic oligomeric or polymeric solubilizing moiety can be a polyethylene glycol, a polypropylene glycol, a copolymer of polyethylene oxide and propylene oxide, a carbohydrate, a detran, or a polyacrylamide. Each solubilizing moiety on a particular molecule can be the same or different.

Each solubilizing moiety can be attached to the dye nucleus by any desired mode. For example, a moiety can be attached to the dye nucleus by bonding a terminal end (e.g., that contains a hydroxyl group), or a non-terminal end of the moiety to the dye nucleus. The point of attachment of the dye nucleus to the solubilizing moiety can be, e.g., a carbon-carbon bond, a carbon-oxygen bond, or a nitrogen-carbon bond. The attachment group for the solubilizing moiety to the dye nucleus can be, e.g., an ester group, a carbonate group, a ether group, a sulfide group, an amino group, an alkylene group, an amide group, a carbonyl group, or a phosphate group.

Specific examples of solubilizing groups are polyethylene glycols, such as —OC(═O)O(CH₂CH₂O)ₙH, —OC(═O)O(CH₂CH₂O)ₙCH₃, —O(CH₂CH₂O)ₙCH₃, and —S(CH₂CH₂O)ₙCH₃, n being an integer between about 10 and about 250; and dextrans, such as —OC(═O)O(dextran).

Each solubilizing moiety can have an absolute molecular weight of from about 500 amu to about 100,000 amu, e.g., from about 1,000 amu to about 50,000 amu or from about 1,500 to about 25,000 amu.

In dyes that include cations of Structure (1), G is H; a moiety that includes at least one amine-, alcohol- or thiol-reactive carboxylic acid group, anhydride group, ester group, or isothiocyanate group, which allows the dyes to be conjugated with another compound that includes an amino group (e.g., a protein), an alcohol group (e.g., a carbohydrate), or a thiol group; or a non-ionic oligomeric or polymeric solubilizing moiety.

If desired, e.g., to improve solubility or biocompatibility, G can include any of the solubilizing moieties discussed above. For example, the solubilizing group can act as a spacer between the dye nucleus and the amine-, alcohol- or thiol-reactive carboxylic acid group, anhydride group, ester group, or isothiocyanate group.

In some embodiments, G is of the form Y′—Ar, in which Y′ is either O or S and Ar is an aromatic moiety or substituted aromatic moiety having the amine-, alcohol- or thiol-reactive carboxylic acid group, anhydride group, ester group, or isothiocyanate group.

In dyes that include cations of Structure (I), R₇, R₈, R₉, R₁₀, R₁₁, R₁₂/R₁₇, R₁₈/R₁₉, R₂₀, R₂₁, and R₂₂ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br, or I, or any two or more of R₇, R₈ and R₉; R₁₀, R₁₁ and R₁₂; and/or R₁₇, R₁₈, R₁₉, R₂₀/R₂₁, and R₂₂ may be bonded together to define a ring that includes between 5 and 12 carbon atoms. The ring that includes between 5 and 12 carbon atoms can be optionally substituted with substituted with one or more F, Cl, Br, I, a C1-C6 straight-chain or branched alkyl, a C1-C6 straight-chain or branched alkoxy, or an aromatic ring having up to 6 carbon atoms, optionally substituted with one or more F, Cl, Br or I. The ring that includes between 5 and 12 carbon atoms can a carbocyclic ring (e.g., a carbocyclic aromatic ring) or a heterocyclic ring (e.g., a heterocyclic aromatic ring). In specific embodiments, R₇, R₈, R₉, R₁₀, R₁₁, R₁₂ and/or R₁₇, R₁₈, R₁₉, R₂₀, R₂₁, and R₂₂ are each H.

Examples of C1-C6 straight-chain or branched alkyl groups include methyl, ethyl, n-propyl, isopropyl, n-pentyl, isopentyl and neopentyl. Examples of C1-C6 straight-chain or branched alkoxy groups include methoxy, ethoxy, n-propoxy, isopropoxy, n-pentoxy, isopentoxy, and neopentoxy.

Examples of aromatic ring systems having up to 6 carbon atoms, optionally substituted with one or more F, Cl, Br, or I, include phenyl groups or substituted phenyl groups (e.g., an attached benzene ring having 1,2-dichloro substitution or 1-chloro-4-fluoro substitution), and heterocyclic aromatic groups or substituted heterocyclic aromatic groups, such as furan, thiophene, imidazole, pyrazole, oxazole, pyridine, and their substituted derivatives.

Some dyes include cations of Structure (XV) shown below.

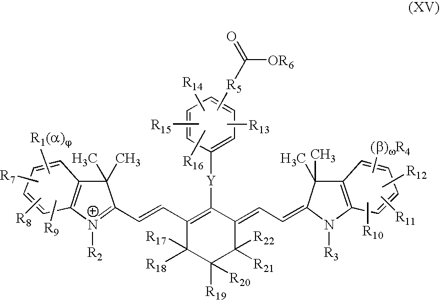

In such dyes, φ and ω are each independently 0 or 1. When φ takes on the value of 0, α is not present and R₁ is bonded directly to the indicated benzene ring, and when ω takes on the value of 0, β is not present and R₄ is bonded directly to the indicated benzene ring. When α and β are present, each can be independently O, S, CH₂, CH₂O, CO₂ or NR′ in which R′ is H or C1-C6 straight-chain or branched alkyl. The C1-C6 straight-chain or branched alkyl groups can be any of those described above in reference to Structure (I).

In the dyes having cations of Structure (XV), R₁, R₂, R₃, and R₄ are each independently PEG moieties defined by (CH₂CH₂O)ₙR″, in which R″ is H or C1-C6 straight-chain or branched alkyl, and n is an integer from 3 to 2,500. The C1-C6 straight-chain or branched alkyl groups those discussed above in reference to Structure (I).

In some embodiments, the PEG chain length and the PEG end group are selected such that the dyes that include the cations of Structure (XV) have a solubility in 10 mM HEPES solution, pH 7.4, of greater than about 10 μM, e.g., water than 25, 50, 75, 100, 125, 150, 200, or even greater than 250 μM.

In the dyes having cations of Structure (XV), Y is S or O; R₅ is (CH₂)ₘ, in which m is an integer from 0 to 8, or a non-ionic oligomeric or polymeric solubilizing moiety and R₆ is H, C1-C6 straight-chain or branched alkyl, or N-succinimidyl. The non-ionic oligomeric or polymeric solubilizing moiety can include any of such moieties described in reference to Structure (I) and the C1-C6 straight-chain or branched alkyl groups can be any of those discussed above in reference to Structure (I).

In the dyes having cations of Structure (XV), R₇, R₈, R₉, R₁₀, R₁₁, R₁₂, R₁₇, R₁₈, R₁₉, R₂₀, R₂₁, and R₂₂ can be any of those described above in reference to Structure (I). R₁₃, R₁₄, R₁₅, and R₁₆ are each independently H, F, Cl, Br, I, C1-C6 straight-chain or branched alkyl, C1-C6 straight-chain or branched alkoxy, or an aromatic ring having up to 6 carbon atoms, optionally substituted with one or more F, Cl, Br or I.

In some embodiments, α and β are O or S and R₁, R₂, R₃ and R₄ are each independently (CH₂CH₂O)ₙR″, in which R″ is H and n is an integer from 10 to 1,000.

In other embodiments, R₇, R₈, R₉, R₁₀, R₁₁, R₁₂, R₁₃, R₁₄, R₁₅, and R₁₆ are each H; α and β are O or S; and R₁, R₂, R₃, and R₄ are each independently (CH₂CH₂O)ₙR″, in which R″ is H and n is an integer from 10 to 1,000.

Some dyes include cations of Structure (VIII) shown below.

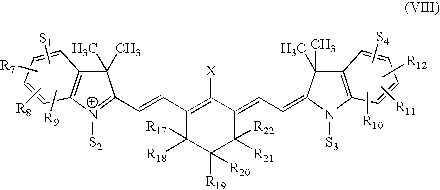

In such dyes, S₁, S₂, S₃, S₄, R₇, R₈, R₉, R₁₀, R₁₁, R₁₂, R₁₇, R₁₈, R₁₉, R₂₀, R₂₁, and R₂₂ are as defined in reference to Structure (I) and X is a good leaving group, such as Cl, Br, I or tosylate.

Any of the cationic dyes described herein that include the cations of Structure (I), (VIII) or (XV) can have nearly any counterion (A⁻), and remain a fluorophoric. For example, the counterion (A⁻) can, e.g., F⁻, Cl⁻, Br⁻, I⁻, ClO₄⁻, or CH₃COO⁻. The dyes can also include mixtures of counterions.

### Absorption and Emission Properties of the Dyes

Generally, the dyes intensely absorb and emit light in the visible and infrared region of the electromagnetic spectrum, e.g., they can emit green, yellow, orange, red light, or near infrared light (“NIR”).

In some embodiments, the dyes emit and/or absorb radiation having a wavelength from about 300 nm to about 1000 nm, e.g., from about 400 nm to about 900 nm, or from about 450 nm to about 850 nm.

In some embodiments the dyes have a maximum excitation and/or a maximum emission, measured in 10 mM HEPES solution, pH 7.4, of from about 525 nm to about 875 nm, e.g., from about 550 nm to about 825 nm, or from about 550 nm to about 800 nm.

### Methods of Preparing the Dyes

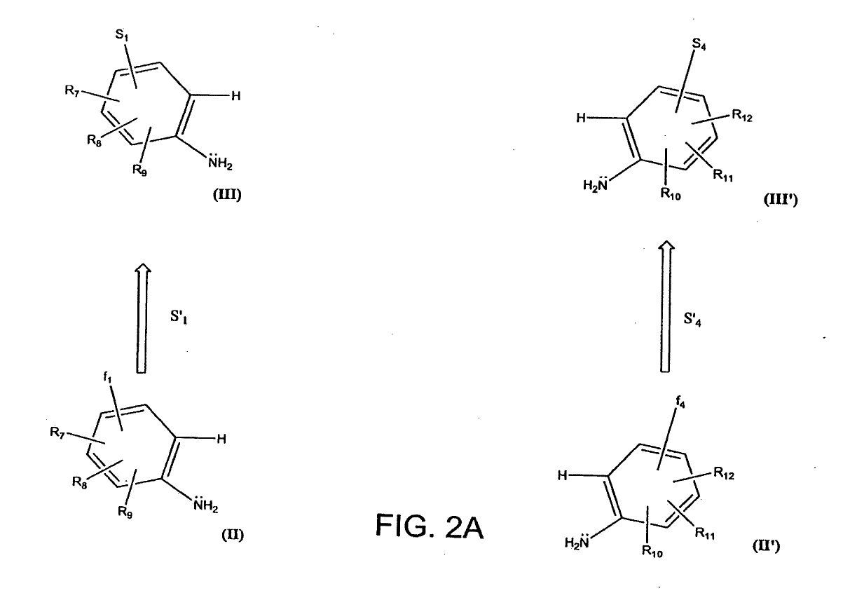

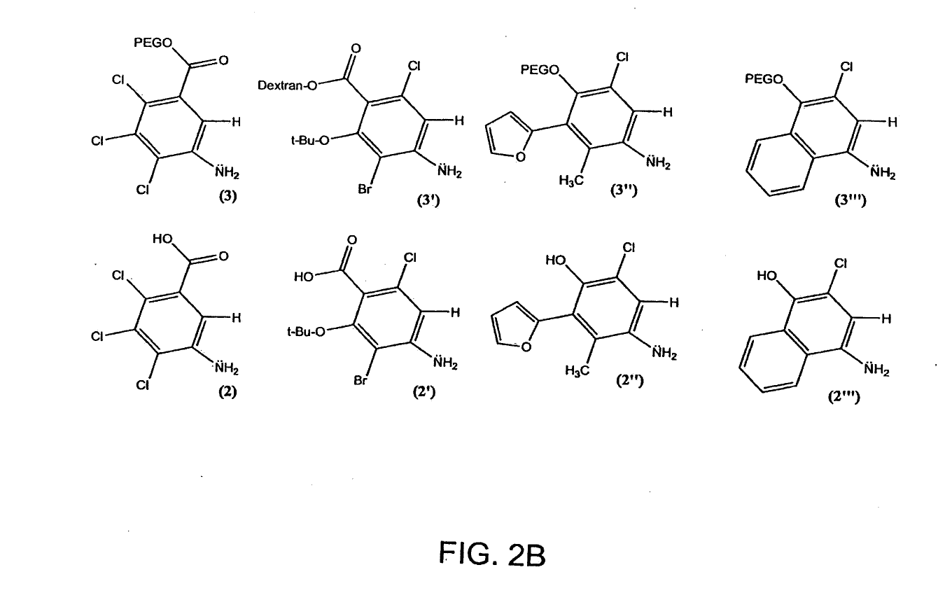

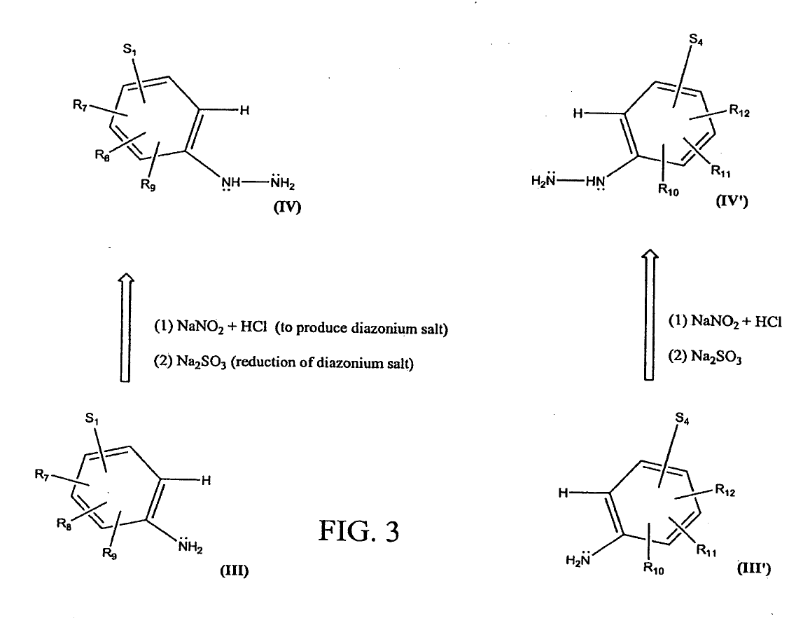

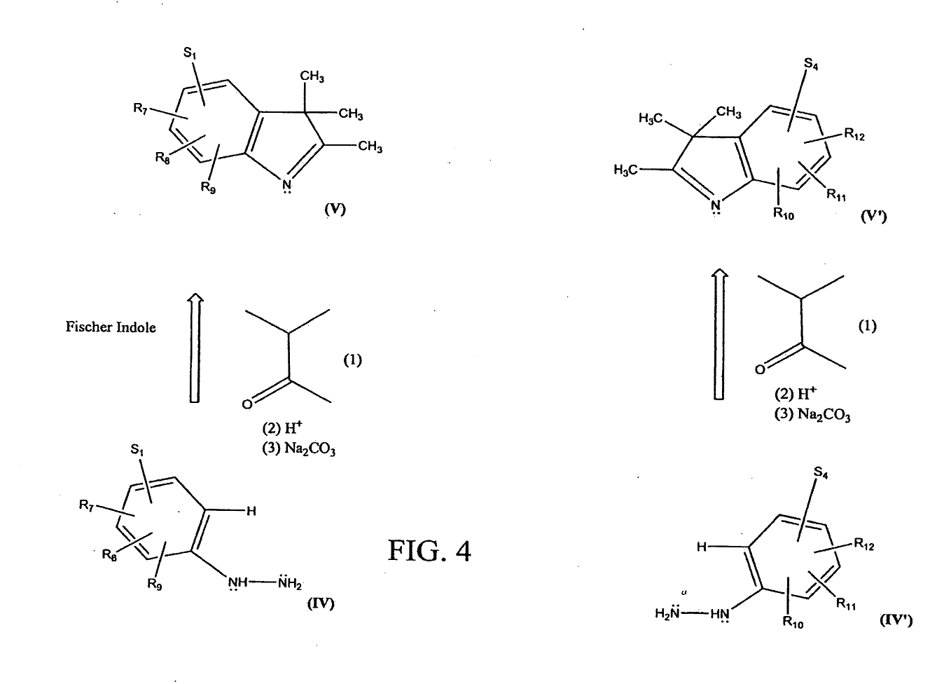

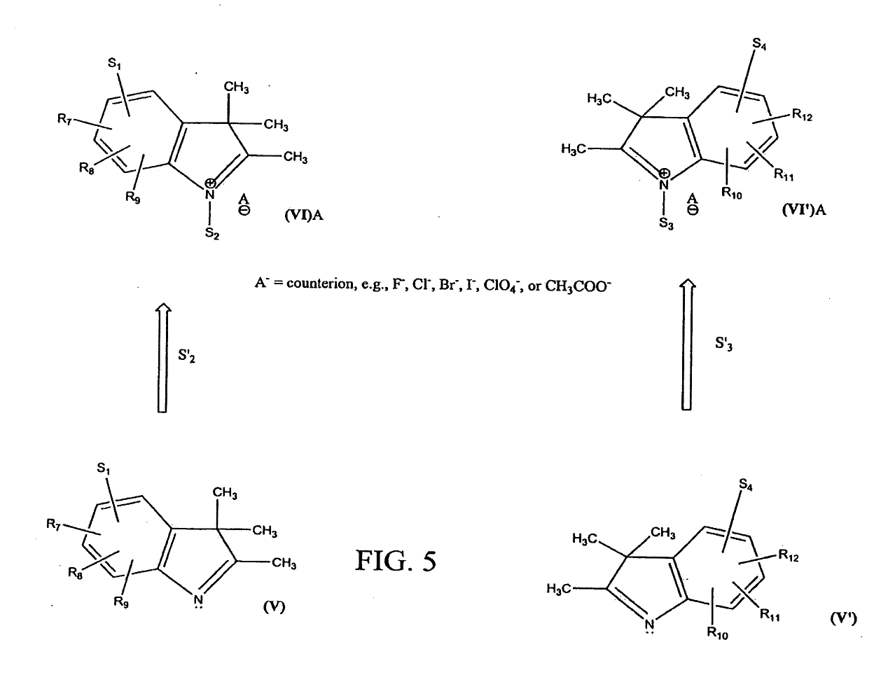

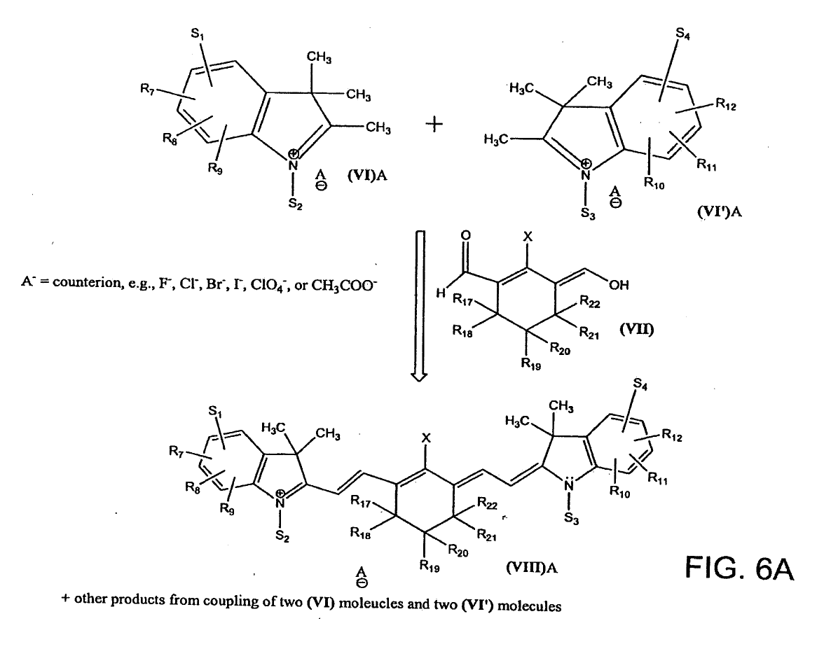

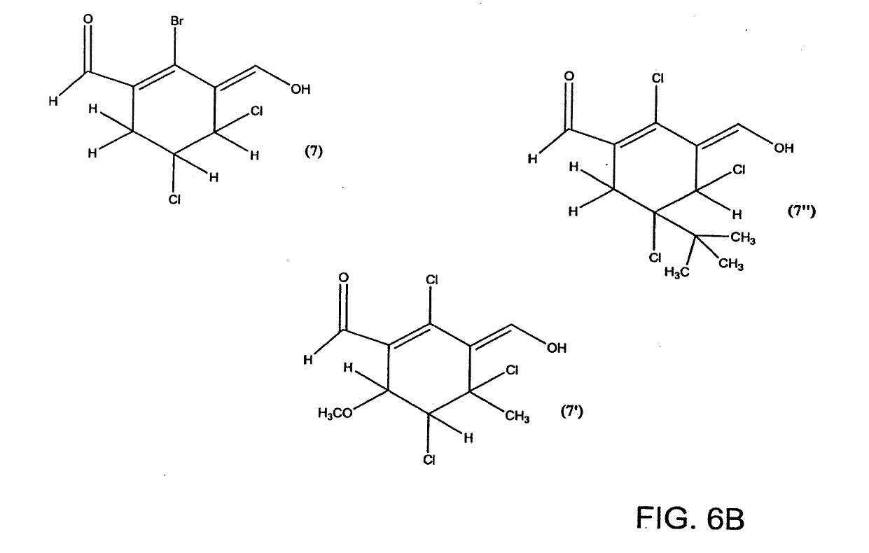

As an overview, FIGS. 2A-6B show that dyes of Structure (XIII)A (FIG. 6A), which include cations of Structure (XIII), can be prepared by first attaching solubilizing arms onto the desired functionalized anilines (FIG. 2A). The resulting anilines having the solubilizing arms are converted to the corresponding hydrazines (FIG. 3), and then the hydrazines are cyclized using methyl isopropyl ketone and the Fischer Indole reaction (FIG. 4). The heterocycles thus formed are then quaternized by attachment of solubilizing arms to the nitrogen atom of each heterocycle (FIG. 5). Finally, the quaternized heterocycles are coupled using the desired hydroxyl methylene cyclohexane (FIG. 6A). This synthetic scheme is described in more detail below.

Referring particularly to FIG. 2A, functionalized anilines of Structures (II) and (II′) are reacted with S′₁ or S′₄, respectively, converting each respective functional group f₁ or f₄ to solubilizing arms S₁ or S₄, to generate anilines of Structures (III) and (III′). Functional groups f₁ and f₄ can be, e.g., a carboxylic acid group (or an ester thereof), or a phenolic oxide group (formed by deprotonating a phenolic hydroxyl group), and S′₁ or S′₄ can be, e.g., α,ω-di-hydroxy polyethylene oxide, dextran, or ethylene oxide. R₇, R₈, and R₉ can be any of the groups described above in reference to Structure (XIII) above. Specific examples of the functionalized anilines prior to attaching solubilizing arms include those shown in FIG. 2A (i.e., compounds 2, 2′, 2″ and 2′″). Specific examples of anilines having attached solubilizing arms are also shown in FIG. 2B (i.e., compound 3, 3′, 3″ and 3′″).

Referring particularly to FIG. 3, anilines having solubilizing arms represented by Structures (III) and (III′) are each reacted with NaNO₂, which produces each respective diazonium salt (not shown). Reduction of each diazonium salt using Na₂SO₃, generates the corresponding hydrazine, represented by Structure (IV) or (IV′).

Referring particularly to FIG. 4, hydrazines of Structures (IV) and (IV') are each cyclized using methyl isopropyl ketone and the Fischer Indole reaction, generating the corresponding heterocycles, represented by Structures (V) and (V′).

Referring particularly to FIG. 5, neutral heterocycles of Structures (V) and (V′) are then each quaternized using S′₂ and S′₃, respectively, generating quaternized heterocyclic compounds of Structures (VI)A and (VI′)A, A being the counterion (e.g., Cl⁻, Br⁻, or I⁻). S′₂ and S′₃ can be, e.g., a solubilizing moiety that includes a good leaving group, such as a halogen. In particular embodiments, S′₂ and/or S′₃ are polyethylene glycols that have a terminal bromide, which can be displaced in a nucleophilic reaction by nitrogen. In other particular embodiments, S′₂ and/or S′₃ is/are ethylene oxide.

Referring particularly to FIG. 6A, quaternized heterocyclic compounds of Structures (VI)A and (VI′)A are coupled using the desired hydroxyl methylene cyclohexene (VII), producing all the possible dyes, which can be separated, e.g., using high performance liquid chromatography (HPLC). R₁₇, R₁₈, R₁₉, R₂₀, R₂₁, and R₂₂ are as defined in reference to Structure (I) (above). Specific examples of the hydroxyl methylene cyclohexenes include those shown in FIG. 6B (i.e., compounds 7, 7′ and 7″).

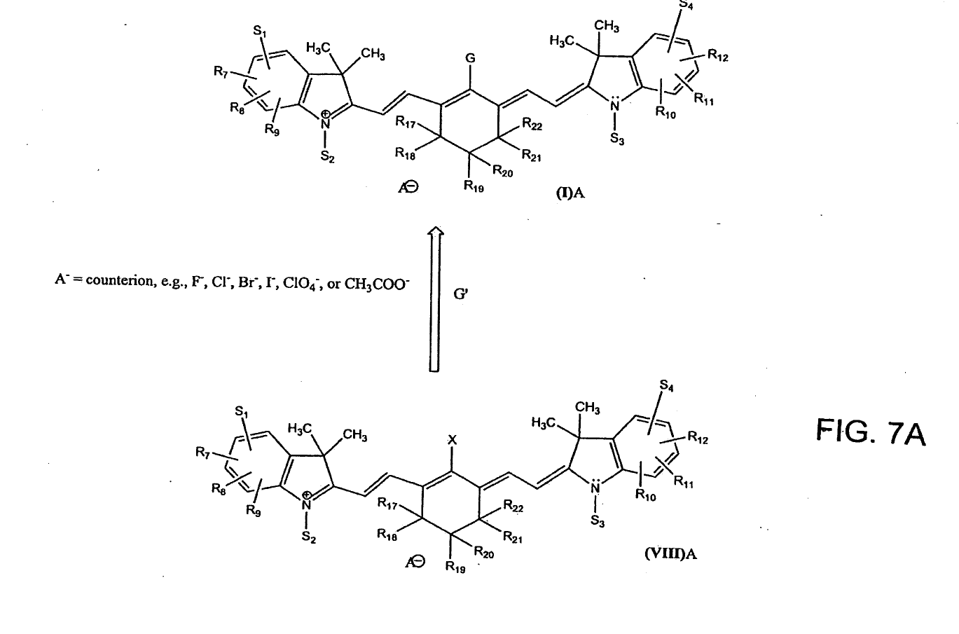

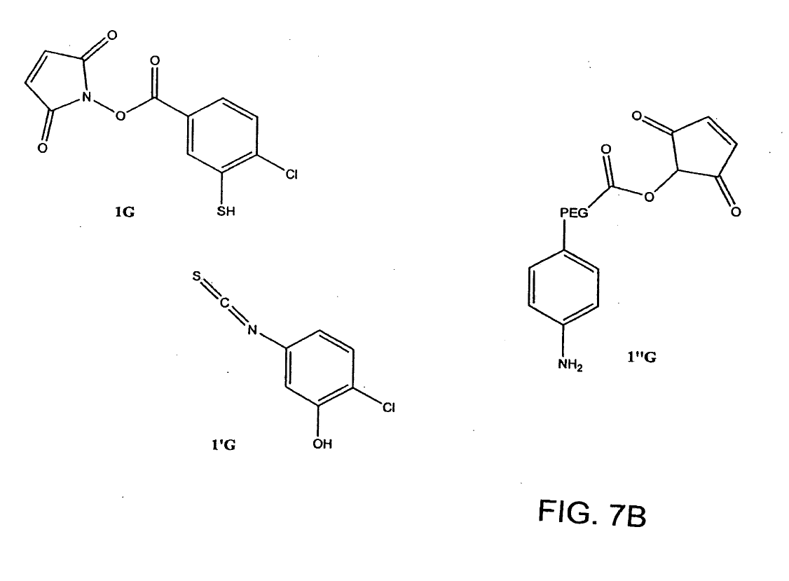

As shown in FIG. 7A, compounds of Structure (I)A, which include cations of Structure (I), can be prepared by reacting compounds of Structure (VIII)A with G′. Specific examples of G′ compounds are those shown in FIG. 7B (i.e., compounds 1G, 1′G, and 1″G).

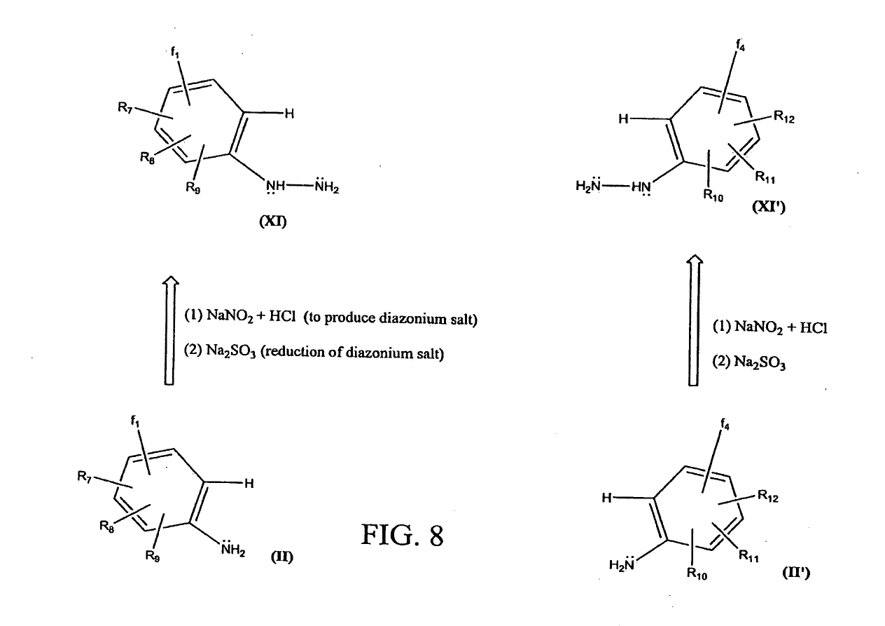

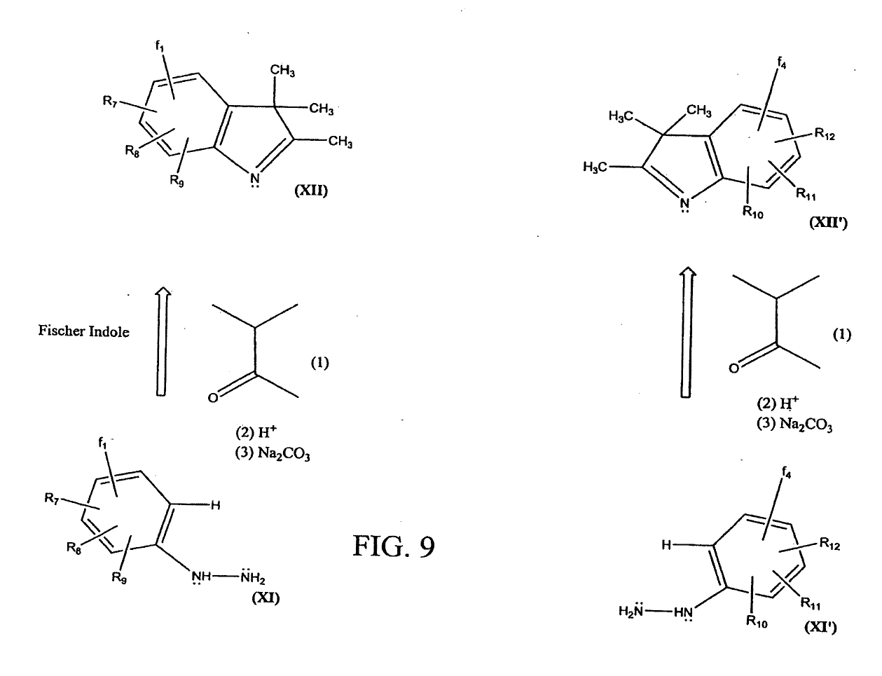

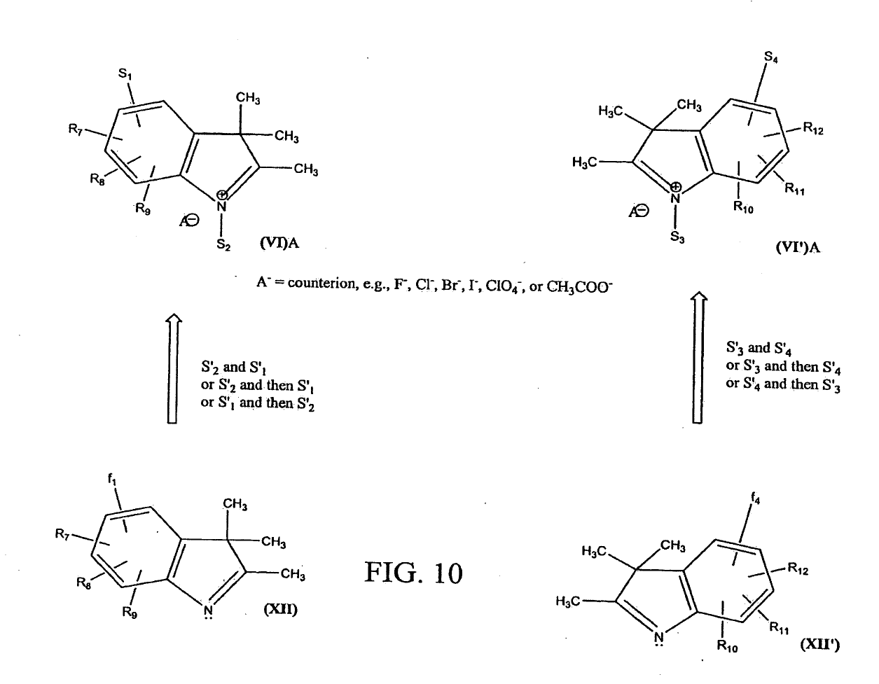

Referring now to FIGS. 8-10, in an alternative synthetic scheme, compounds of Structures (VI)A and (VI′)A (FIG. 6A or FIG. 10) can be prepared by forming hydrazines from the corresponding functionalized anilines (FIG. 8), without first attaching solubilizing arms (as was shown in FIGS. 2A-8). The hydrazine Structures (XI) and (XI′), without solubilizing arms, are cyclized using methyl isopropyl ketone and the Fischer Indole reaction (FIG. 9). The cyclized products of Structures (XII) and (XII′) are then concurrently, or in a step-wise fashion, functionalized and quaternized with solubilizing arms, generating compounds Structures (VI)A and (VI′)A (FIG. 10). Compounds of Structures (VI) and (VI′)A can then be coupled as described above.

When desired and/or necessitated to effect any chemical transformation, any of the functional groups in any of the synthetic schemes shown herein can be protected by protecting groups, which can be removed in a later step to produce the desired compound.

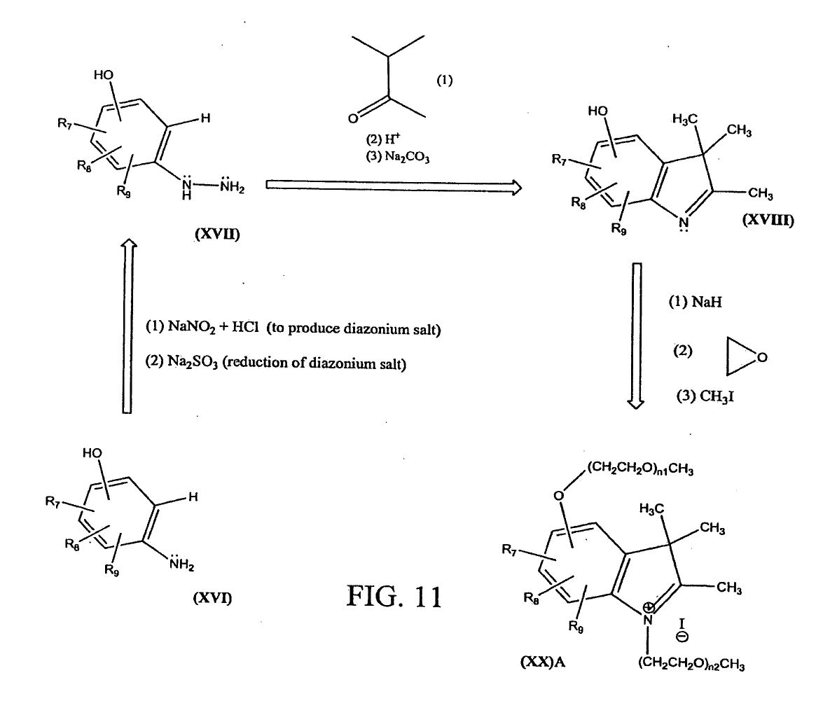

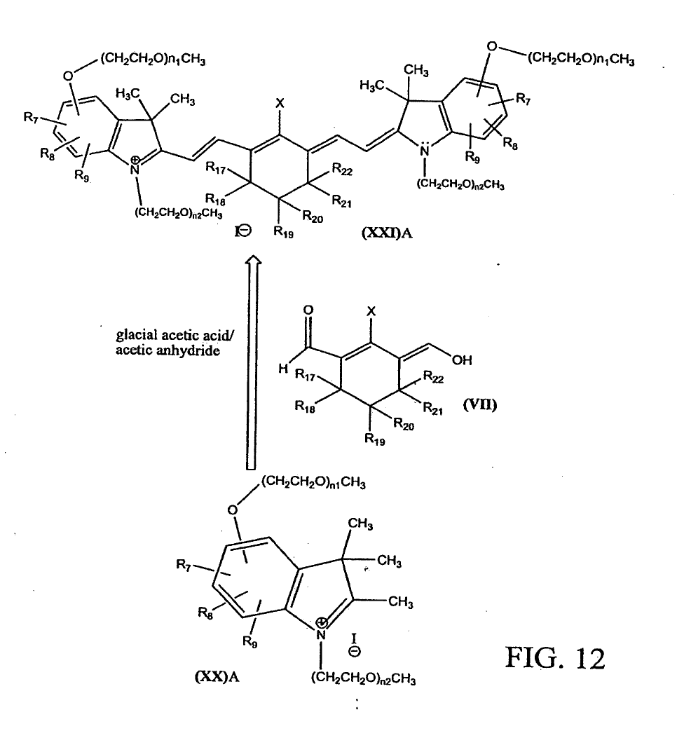

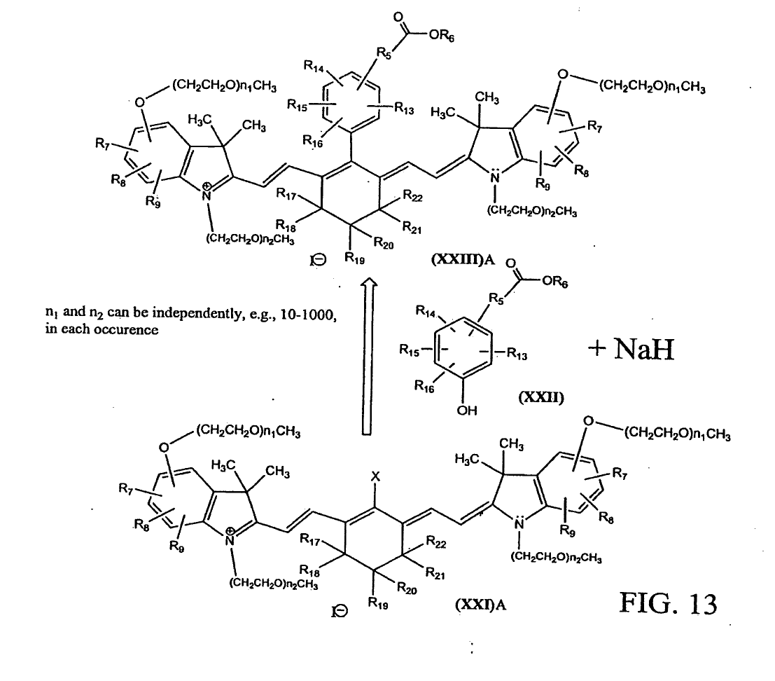

FIGS. 11-13 show that to make dyes of Structure (XXI)A (FIG. 12), and dyes of Structure (XXIII)A of (FIG. 13), hydroxyl substituted anilines of Structure (XVI) are converted to their corresponding hydrazines of Structure (XVII), and cyclized to produce compounds of Structure (XVIII). The heterocycles thus formed are then reacted with sodium hydride to produce the corresponding phenoxide (not shown), and the phenoxide is reacted with ethylene oxide. Living, polymeric side chains are quenched with methyl iodide to produce quaternized salts of Structure (XX)A having PEG solubilizing arms that are terminated with a methyl group. Compounds of Structure (XX)A can then be converted to dyes of Structure (XXI)A by reaction with methylene cyclohexenes of Structure (VII), as shown in FIG. 12. Dyes of Structure (XXI)A can be converted to dyes of Structure (XXIII)A by reaction of dyes of Structure (XXI)A with the phenolic compounds of Structure (XXII), as shown in FIG. 13.

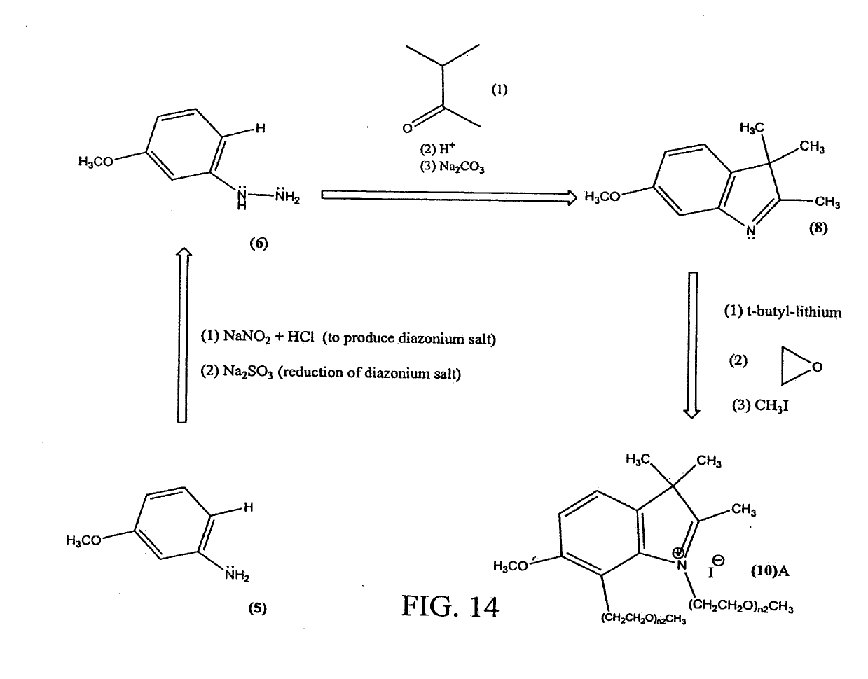

A specific example of a synthetic reaction scheme is shown in FIG. 14. Compound (10)A can be made by converting m-methoxyaniline (5) to its hydrazine (6), and then cyclizing the hydrazine to produce heterocycle (8). Heterocycle (8) can then be metallated in the alpha position to the ring and the methoxy group with t-butyl-lithium, and then the metallated species can be reacted with ethylene oxide. The living polymer chain can be quenched after growing to a desired length with methyl iodide, producing compound (10)A having PEG groups terminated with methyl groups. In some embodiments, each PEG chain is allowed to grow such that n₁ and n₂ are each independently between about 4 and about 2,500, e.g., from about 10 to about 1000, or from about 15 to about 500.

Other synthetic schemes that can be applied to making dyes are described in Frangioni et al., U.S. Provisional Patent Application Ser. No. 60/835,407, filed on Aug. 3, 2006, the entire contents of which is incorporated herein by reference.

### Dye Conjugates

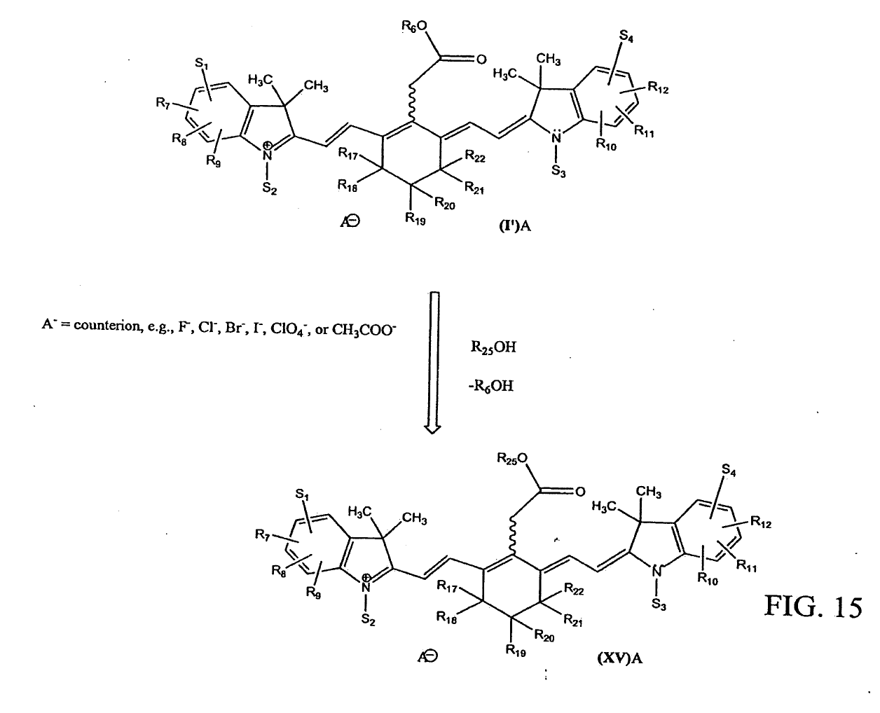

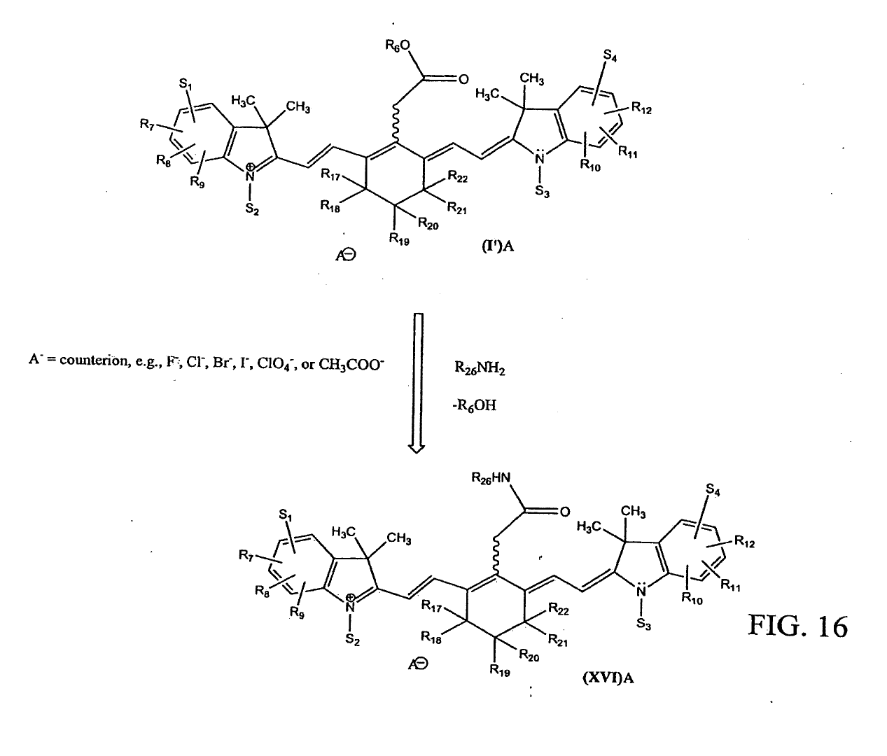

Any of the dyes described herein, e.g., dyes that include cations of Structures (I), (VIII), or (XV), can be reacted with other compounds, e.g., oligomers or polymers that contain amine-, alcohol-, or thiol-groups, such as targeting ligands (e.g., small molecule peptides, proteins, protein fragments, peptides, antibodies, carbohydrates, or antigens), to form conjugates. The conjugates can target the dye to specific tissues, and can be used for real time surgical guidance for identifying tumors, and other abnormal tissues. For example, FIGS. 15 and 16 show, respectively, reaction of dyes of Structure (I′)A with a hydroxyl-containing moiety, and an amine-containing moiety to form a conjugate.

In a typical conjugation procedure, all of the following steps can be performed under reduced light conditions in dimethyl sulfoxide (DMSO) at room temperature. In one procedure, each 50 μL reaction contains 20 mM triethylamine (TEA), 1 mM of the desired ligand, and 1 mM of the desired dye, which are added in the mentioned order. To effect the conjugation, the reaction mixture is constantly agitated for 18 hours in the dark. Additional general details for conjugation of dyes is discussed in Frangioni et al., Molecular Imaging, vol. 1(4), 354-364 (2002).

Specific proteins, protein fragments, peptides, antibodies, carbohydrates, or antigens that can be used to form the new conjugates are described, e.g., in Frangioni et al. in “MODIFIED PSMA LIGANDS AND USES RELATED THERETO”, WO 02/098885, filed on Feb. 7, 2002 (now issued as U.S. Pat. No. 6,875,886). A specific targeting ligand is the RGD peptide, which specifically binds to alphₐᵥβ₃ integrin. It is known that this integrin is overexpressed by various tumors, and thus, these RGD targeting peptides enable the dyes to preferentially label tumors that overexpress these integrins. Other targeting ligands include melanocyte stimulating hormone (MSH), which targets melanoma cells, or bombesin, somatostatin, or Sandostatin™ (synthetic), which target somatostatin receptors.

### Applications

The dyes and dye conjugates, e.g., dye-biomolecule conjugates, can be used for, e.g., optical tomographic, endoscopic, photoacoustic, and sonofluorescent applications for the detection, imaging, and treatment of tumors and other abnormalities.

The dyes and dye conjugates can also be used for localized therapy. This can be accomplished, e.g., by attaching a porphyrin or other photodynamic therapy agent to a bioconjugate; directing the conjugates to a desired target site, or allowing the conjugates to accumulate selectively in the target site; shining light of an appropriate wavelength to activate the agent. Thus, the new conjugates can be used to detect, image, and treat a section of tissue, e.g., a tumor.

In addition, the dyes and conjugates can be used to detect the presence of tumors and other abnormalities by monitoring the blood clearance profile of the conjugates, for laser assisted guided surgery for the detection of small micrometastases of, e.g., somatostatin subtype 2 (SST-2) positive tumors, and for diagnosis of atherosclerotic plaques and blood clots.

### Dyes and Dye Conjugate Compositions

The dyes and dye conjugates can be formulated into diagnostic and therapeutic compositions for enteral or parenteral administration. Generally, these compositions contain an effective amount of the dye or dye conjugate, along with conventional pharmaceutical carriers and excipients appropriate for the type of administration contemplated. For example, parenteral formulations include the dye or dye conjugate in a sterile aqueous solution or suspension. Parenteral compositions can be injected directly into a subject at a desired site, or mixed with a large volume parenteral composition for systemic administration. Such solutions can also contain pharmaceutically acceptable buffers and, optionally, electrolytes, such as sodium chloride.

Formulations for enteral administration, in general, can contain liquids, which include an effective amount of the desired dye or dye conjugate in aqueous solution or suspension. Such enteral compositions can optionally include buffers, surfactants, and thixotropic agents. Compositions for oral administration can also contain flavoring agents, and other ingredients for enhancing their organoleptic qualities.

Generally, the diagnostic compositions are administered in doses effective to achieve the desired signal strength to enable detection. Such doses can vary, depending upon the particular dye or dye conjugate employed, the organs or tissues to be imaged, and the imaging equipment being used. For example, Zeheer et al., Nature Biotechnology, 19, 1148-1154 (2001) uses 0.1 μmol/kg as a dose for IRDye78 conjugates in vivo. The diagnostic compositions can be administered to a patient systemically or locally to the organ or tissue to be imaged, and then the patient is subjected to the imaging procedure.

### OTHER EMBODIMENTS

A number of embodiments have been described. Nevertheless, it will be understood that various modifications may be made without departing from the spirit and scope of the invention. Other embodiments are within the scope of the following claims.

1. A compound comprising a cation of Structure (I): 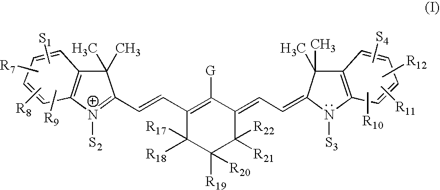
wherein
S₁, S₂, S₃, and S₄ are each independently a non-ionic oligomeric or polymeric solubilizing moiety;
G is H or a moiety that includes at least one amine-, alcohol- or thiol-reactive carboxylic acid group, anhydride group, ester group, or isothiocyanate group; and
R₇, R₈, R₉, R₁₀, R₁₁, R₁₂, R₁₇, R₁₈, R₁₉, R₂₀, R₂₁, and R₂₂ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br, or I, or any two or more of R₇, R₈, and R₉ and/or any two or more of R₁₀, R₁₁, and R₁₂; and/or R₁₇, R₁₈, R₁₉, R₂₀, R₂₁, and R₂₂ may be bonded together to define a ring that includes between 5 and 12 carbon atoms, wherein the ring is optionally substituted with one or more F, Cl, Br, or I.

2. A compound of claim 1, wherein each of S_{I}, S₁, S₃, and S₄ is selected such that the compound has a solubility in 10 mM HEPES solution, pH 7.4, of greater than about 10 μM.

3. A compound of claim 1, wherein each of S₁, S₂, S₃, and S₄ is selected such that the compound that comprises cation of Structure (I) has a solubility in 10 mM HEPES solution, pH 7.4, of greater than about 50 μM.

4. A compound of claim 1, wherein S₁, S₂, S₃, and S₄ are each independently selected from the group consisting of a polyethylene glycol, a polypropylene glycol, a copolymer of polyethylene oxide and propylene oxide, a carbohydrate, a dextran, and a polyacrylamide.

5. A compound of claim 1, wherein G is Y′—Ar, wherein Y′ is O or S and Ar is an aromatic moiety or substituted aromatic moiety having an amine-, alcohol-, or thiol-reactive carboxylic acid group, anhydride group, ester group, or isothiocyanate group.

6. A compound of claim 1, wherein R₇, R₈, R₉, R₁₀, R₁₁, and R₁₂ are each H.

7. A compound of claim 1, wherein R₁₇, R₁₈, R₁₉, R₂₀, R₂₁, and R₂₂ are each H.

8. A compound of claim 1, comprising cations of Structure (XV) 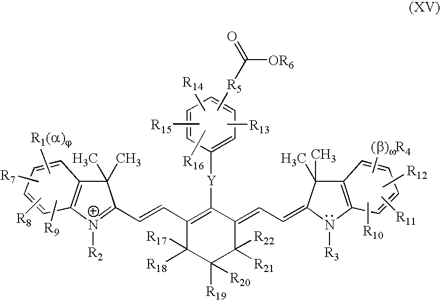
wherein
φ and ω are each independently 0 or 1;
α and β are each independently O, S, CH₂, CH₂O, CO₂, or NR′ in which R′ is H or C1-C6 straight-chain or branched alkyl;
R₁, R₂, R₃, and R₄ are each independently (CH₂CH₂O)ₙR″ in which R″ is H or C1-C6 straight-chain or branched alkyl, and n is an integer from 4 to 2,500;
Y is S or O;
R₅ is (CH₂)ₘ, m being an integer from 0 to 8, or a non-ionic oligomeric or polymeric solubilizing moiety;
R₆ is H, C1-C6 straight-chain or branched alkyl, or N-succinimidyl; and
R₇, R₈, R₉, R₁₀, R₁₁, R₁₂, R₁₃, R₁₄, R₁₅, R₁₆, R₁₇, R₁₈, R₁₉, R₂₀, R₂₁, and R₂₂ are each independently H, F, Cl, Br, I, C1-C6 straight-chain or branched alkyl, C1-C6 straight-chain or branched alkoxy, or an aromatic ring having up to 6 carbon atoms, optionally substituted with one or more F, Cl, Br, I.

9. A compound of claim 8, wherein φ and ω are each 1.

10. A compound of claim 8, wherein α and β are each independently O or S.

11. A compound of claim 8, wherein R₁, R₂, R₃, and R₄ are each independently (CH₂CH₂O)ₙR″ in which R″ is H and n is an integer from 8 to 1,000.

12. A compound of claim 8, wherein Y is O.

13. A compound of claim 8, wherein R₅ is (CH₂)ₘ, and in is an integer from 0 to 8.

14. A compound of claim 8, wherein R₆ is H or N-succinimidyl.

15. A compound of claim 8, wherein R₇, R₈, R₉, R₁₀, R₁₁, R₁₂, R₁₃, R₁₄, R₁₅, and R₁₆ are each H.

16. A compound of claim 8 wherein R₁₇, R₁₈, R₁₉, R₂₀, R₂₁, and R₂₂ are each H.

17. A compound of claim 1, further comprising an anion selected from the group consisting of F⁻, Cl⁻, Br⁻, I⁻, ClO₄⁻, CH₃COO⁻, and mixtures thereof.

18. A reaction product of claim 1, and an amino-, or hydroxyl-, or thiol-containing moiety.

19. The reaction product of claim 18, wherein the amino-containing moiety is a small molecule peptide, a protein, a polypeptide, an antibody, or an antigen.

20. The reaction product of claim 18, wherein the hydroxyl-containing group is a carbohydrate.

21. A compound of Structure (V): 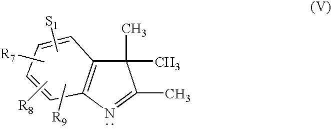
wherein
S₁ is a non-ionic oligomeric or polymeric solubilizing moiety; and
R₇, R₈, R₉ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br, or any two or more of R₇, R₈ and R₉ may be bonded together to define a ring that includes between 5 and 12 carbon atoms, wherein the ring is optionally substituted with one or more F, Cl, Br, or I.

22. A compound of claim 21, wherein S₁ is selected such that the compound has a solubility in 10 mM HEPES solution, pH 7.4, of greater than about 10 μM.

23. A compound comprising a cation of Structure (VI) 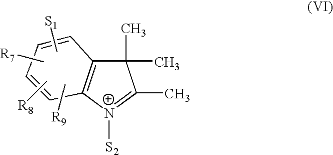
wherein
S₁ and S₂ are each independently a non-ionic oligomeric or polymeric solubilizing moiety; and
R₇, R₈, R₉, are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br, or I, or any two or more of R₇, R₈ and R₉, may be bonded together to define a ring that includes between 5 and 12 carbon atoms, wherein the ring is optionally substituted with one or more F, Cl, Br, or I.

24. A compound of claim 23, wherein S₁ and S₂ are each selected such that the compound has a solubility in 10 mM HEPES solution, pH 7.4, of greater than about 10 μM.

25. A compound of claim 23, further comprising an anion selected from the group consisting of F, Cl⁻, Br⁻, I⁻, ClO₄⁻, and CH₃COO⁻.

26. A compound comprising a cation of Structure (VIII): 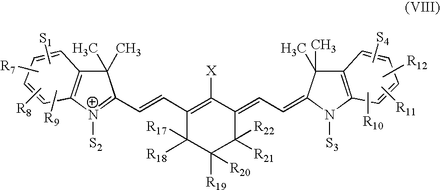
wherein
S₁, S₂, S₃, and S₄ are each independently a non-ionic oligomeric or polymeric solubilizing moiety;
X is a Cl, Br, I, or tosylate; and
R₇, R₈, R₉, R₁₀, R₁₁, R₁₂, R₁₇, R₁₈, R₁₉, R₂₀, R₂₁, and R₂₂ are each independently H, F, Cl, Br, I, C1-C6 straight-chain or branched alkyl, C1-C6 straight-chain or branched alkoxy, an aromatic ring having up to 6 carbon atoms, optionally substituted with one or more of F, Cl, Br, or I, or any two or more of R₇, R₈, and R₉ and/or any two or more of R₁₀, R₁₁, and R₁₂; and/or any two or more of R₁₇, R₁₈, R₁₉, R₂₀, R₂₁, and R₂₂ may be bonded together to define a ring that includes between 5 and 12 carbon atoms, wherein the ring is optionally substituted with one or more F, Cl, Br, or I.

27. A compound of claim 26, wherein each of S₁, S₂, S₃, and S₄ is selected such that the compound has a solubility in 10 mM HEPES solution, pH 7.4, of greater than about 10 μM.

28. A reaction product of compounds of claim 26 and an amino-, hydroxyl-, or a thiol-containing moiety.

29. A dye comprising:
a positively charged nitrogen-containing dye core comprising a conjugated heptamethine or substituted heptamethine system;
one or more non-ionic solubilizing molecular arms bonded to the dye core; and
optionally, one or more functionalizable molecular arms bonded to the dye core,
wherein the one or more functionalizable molecular arms each comprise an amine-, alcohol-, or thiol-reactive carboxylic acid group, anhydride group, ester group, or isothiocyanate group.

30. A dye of claim 29, wherein the positively charged nitrogen-containing dye core has a single positive charge.

31. A dye of claim 29, wherein the one or more solubilizing molecular arms are selected such that the dye has a solubility in 10 mM HEPES solution, pH 7.4, of greater than about 10 μM.

32. A dye of claim 29, wherein the one or more solubilizing molecular arms are selected such that the dye has a solubility in 10 mM HEPES solution, pH 7.4, of greater than about 25 μM.

33. A dye of claim 29, wherein the one or more solubilizing molecular arms are selected such that the dye has a solubility in 10 mM HEPES solution, pH 7.4, of greater than about 50 μM.

34. A dye of claim 29, wherein the one or more solubilizing molecular arms are also functionalized with an amine-, alcohol-, or thiol-reactive carboxylic acid group, anhydride group, ester group, or isothiocyanate group.

35. A dye of claim 29, wherein the dye has a maximum excitation and/or a maximum emission, measured in 10 mM HEPES solution, pH 7.4, of from about 525 nm to about 875 nm.

36. A dye of claim 29, wherein the dye has a maximum excitation and/or a maximum emission, measured in 10 mM HEPES solution, pH 7.4, of from about 550 nm to about 825 nm.

37. A dye of claim 29, wherein the dye has a maximum excitation and/or a maximum emission, measured in 10 mM HEPES solution, pH 7.4, of from about 550 nm to about 800 nm.

38. A dye of claim 29, wherein the one or more non-ionic solubilizing molecular arms and/or the one or more functionalizable molecular arms are bonded to the heptamethine system of the dye.

39. A method of making a compound, the method comprising:
attaching a non-ionic solubilizing moiety to a functionalized aniline having a hydrogen atom ortho to an amino group of the aniline to provide an aniline having a solubilizing arm;
converting the aniline having the solubilizing arm to its corresponding hydrazine; and
cyclizing the hydrazine with a cyclizing moiety to form a nitrogen-containing, fused heterocyclic ring having points of fusion at a point of attachment of the hydrazine and at a location ortho to the point of attachment of the hydrazine.

40. The method of claim 39, further comprising quaternizing a nitrogen atom of the nitrogen-containing fused heterocyclic ring with a quaternizing moiety to provide a quaternized nitrogen-containing compound.

41. The method of claim 40, wherein the quaternizing moiety comprises an amine-, alcohol-, or thiol-reactive group.

42. The method of claim 41, wherein the amine-, alcohol-, or thiol-reactive group is a carboxylic acid group, anhydride group, ester group, or isothiocyanate group.

43. The method of claim 40, further comprising coupling quaternized nitrogen-containing compounds with a coupling moiety to provide a nitrogen-containing core bearing a positive charge and comprising a conjugated heptamethine system bridging fused heterocyclic rings.

44. A method of making a compound, the method comprising:
converting a functionalized aniline having a hydrogen atom ortho to an amino group of the aniline to its corresponding functionalized hydrazine; and
cyclizing the functionalized hydrazine with a cyclizing moiety to form a nitrogen-containing, fused heterocyclic ring having points of fusion at a point of attachment of the hydrazine and at a location ortho to the point of attachment of the hydrazine to provide a functionalized fused heterocyclic compound; and
attaching a non-ionic solubilizing moiety to the functionalized fused heterocyclic compound to provide a functionalized fused heterocyclic compound having a solubilizing arm.

45. The method of claim 44, further comprising quaternizing a nitrogen atom of the functionalized fused heterocyclic compound having a solubilizing arm with a quaternizing moiety to provide a quaternized nitrogen-containing compound.

46. The method of claim 45, further comprising coupling quaternized nitrogen-containing compounds with a coupling moiety to provide a nitrogen-containing core bearing a positive charge and comprising a conjugated heptamethine system bridging fused heterocyclic rings.

47. The method of claim 39, wherein the non-ionic solubilizing moiety is polymeric.

48. The method of claim 39, wherein the cyclizing moiety is methyl isopropyl ketone.

49. A method of making a conjugate, the method comprising:
providing a compound of claim 1, wherein the compound has at least one amine-, alcohol-, or thiol-reactive carboxylic acid group, anhydride group, ester group, or isothiocyanate group; and
reacting the provided compound with a moiety that includes an amino, hydroxyl or thiol group to provide a conjugate.

50. A method of imaging or treating abnormal tissue and/or cells, the method comprising:
administering to a subject a conjugate comprising a reaction product of claim 1 and a amino- hydroxyl- or thiol-containing moiety, wherein the amino- hydroxyl- or thiol-containing moiety is capable of binding with a complementary site on the abnormal tissue and/or cells; and
irradiating the conjugate at a wavelength that the conjugate absorbs radiation.

51. The method of claim 50, further comprising detecting and/or quantifying absorbed and/or emitted radiation.

52. (canceled)
